(19) United States
(12) Patent Application Publication
(10) Pub. No.: US 2017/0157186 A1
(43) Pub. Date: Jun. 8, 2017

(54) PHAGE TO TREAT BACTERIA ON SKIN

(71) Applicant: Smart Phage, Inc., Houston, TX (US)

(72) Inventor: Jacob Shiach, Houston, TX (US)

(21) Appl. No.: 15/368,224

(22) Filed: Dec. 2, 2016

Related U.S. Application Data
(60) Provisional application No. 62/262,051, filed on Dec. 2, 2015.

(51) Int. Cl. A61K 35/76; A61K 9/70

(52) U.S. Cl. CPC A61K 35/76; A61K 9/70; C12N 2795/00032

(57) ABSTRACT

Methods and compositions having phage to prevent, inhibit or treat bacterial infection, e.g., on the dermis, are provided.

### CROSS-REFERENCE TO RELATED APPLICATIONS

This application claims the benefit of the filing date of U.S. application Ser. No. 62/262,051, filed on Dec. 2, 2015, the disclosure of which is incorporated by reference herein.

### BACKGROUND

A number of bacteria pose problems when their growth on skin is not contained. For instance, Staphylococcus aureus is a dangerous pathogen responsible for a variety of infections including skin abscesses. Staphylococcus epidermidis is a common skin and mucous commensal of healthy humans, and is involved in balancing epithelial microbiota. However, S. epidermidis can form biofilms favoring adhesion to host tissues and increasing resistance to antibiotics.

Propionibacterium acnes is a universal inhabitant of human skin and is found at high population densities on the fat-rich areas of the face, scalp, and upper trunk. P. acnes can form biofilms and has been implicated in the pathogenesis of inflammatory acne, as treatments that reduce the numbers of P. acnes cells on the skin are therapeutic and the failure of such therapies has been associated with the emergence of resistance in P. acnes. Acne vulgaris is the most common disease of skin in adolescents and affects approximately 80% of individuals at some stage in their lives. This disease can be extremely painful and can lead to numerous psychological problems. Inflammatory lesions give rise to significant scar formation in 30% of patients.

### SUMMARY

The invention provides for packaging of phage or a plurality of distinct phage, and delivery of phage or a plurality of distinct phage, for skin care, e.g., to rebalance skin microbiomes for cosmetic purposes. In one embodiment, the phage is delivered using a natural fiber substrate, e.g., cotton, eucalyptus, or biocellulose. The phage may be resuspended in buffer or gel (e.g., pH of about 4.0 to about 8.0) at a concentration of about >10⁴ VLP/mL, e.g., about >10⁶ VLP/mL. In one embodiment, the phage may be resuspended in a gel (e.g., pH of about 4.0 to about 8.0) at a concentration of about >10⁶ VLP/mL. In one embodiment, the phage are present at a concentration of about 10⁷ VLP/mL to about 10⁸ VLP/mL. The phage may be resuspended in buffer or gel (e.g., pH of about 4.0 to about 8.0) at a concentration of about >10⁴ plaque forming units (PFU)/mL, e.g., about >10⁶ PFU/mL. In one embodiment, the phage may be resuspended in a gel (e.g., pH of about 4.0 to about 8.0) at a concentration of about >10⁶ PFU/mL. In one embodiment, the phage are present at a concentration of about 10⁷ PFU/mL to about 10⁸ PFU/mL. The phage or a phage cocktail may include at least two of Propionibacterium acnes targeting phage, Staphylococcus epidermidis targeting phage, and/or Staphylococcus aureus targeting phage. In one embodiment, the buffer contains 0%-100%%, or any integer or range of integers, of Propionibacterium acnes targeting phage, 0%-100%, or any integer or range of integers, of Staphylococcus epidermidis targeting phage, and/or 0%-100%%, or any integer or range of integers, of Staphylococcus aureus targeting phage, e.g., at least 1% of Propionibacterium acnes targeting phage, Staphylococcus epidermidis targeting phage, or Staphylococcus aureus targeting phage. As used herein, “specific for,” “with specifity” or “targeting” means that the phage binds isolates of that species of bacteria via receptors on the surface of the bacteria, which receptors are not found on all types of bacteria. For instance, if a phage is specific for P. acnes, it infects (and lyses) P. acnes but not, for example, S. aureus or S. epidermidis.

In one embodiment, the invention provides a composition for topical application. The composition includes a biocompatible substrate comprising a natural fiber coated or embedded with one or more isolated phage with specificity for Propionibacterium acnes, Staphylococcus epidermidis or Staphylococcus aureus, or a combination of phage, in an amount effective to inhibit or treat Propionibacterium acnes, Staphylococcus epidermidis or Staphylococcus aureus present on human dermis. In one embodiment, the fiber comprises cotton, eucalyptus or biocellulose or a combination thereof. In one embodiment, the one or more isolated phage are in an aqueous buffer. In one embodiment, the one or more isolated phage are in a gel. In one embodiment, the phage are embedded in the substrate. In one embodiment, the phage have a titer of at least 10⁶ VLP/mL or at least 10⁶ PFU/mL. In one embodiment, the fiber is coated or embedded with one or more strains of phage specific for P. acnes. In one embodiment, the fiber is coated or embedded with one or more strains of phage specific for S. aureus. In one embodiment, the fiber is coated or embedded with one or more strains of phage specific for S. epidermis. In one embodiment, the fiber is embedded with phage specific for P. acnes, phage specific for S. aureus, and phage specific for S. aureus. Phage selected for use in the compositions and methods are stable in solutions at a range of pH, e.g., from about pH 4.0 to about pH 8.0, and with various buffers, e.g., phosphate buffered saline (PBS) or Tris-HCl/NaCl/MgSO₄, and in packaging stored at ambient temperatures. In one embodiment, the solutions have a pH in the range from about 6.8 to 7.8.

Further provided is a method to prepare a substrate to treat or inhibit bacterial growth on skin. The method includes providing a biocompatible substrate comprising a natural fiber and an aqueous buffer or gel comprising one or more isolated phage specific for Propionibacterium acnes, Staphylococcus epidermidis or Staphylococcus aureus or a combination of phage, in an amount effective to inhibit or treat Propionibacterium acnes, Staphylococcus epidermidis or Staphylococcus aureus growth on human dermis. In one embodiment, the aqueous buffer or gel is brought into contact with the substrate. In one embodiment, the fiber comprises cotton, eucalyptus, bamboo fiber, or biocellulose. In one embodiment, the one or more phage are in an aqueous buffer. In one embodiment, the one or more isolated phage are in a gel. In one embodiment, the gel comprises polyvinyl alcohol. In one embodiment, the buffer is applied by immersing the substrate in the buffer. In one embodiment, the substrate is dry.

Also provided is a method to prevent, treat or inhibit bacterial growth on skin. The method includes applying to dermis of a human a biocompatible substrate comprising a natural fiber coated or embedded with one or more isolated phage with lytic activity specific for Propionibacterium acnes, Staphylococcus epidermidis or Staphilococcus aureus, or a combination thereof, in an amount effective to inhibit or treat Propionibacterium acnes, Staphylococcus epidermidis or Staphilococcus aureus.

Further provided is a method to prevent, inhibit or treat n awn of the dermis by Micrococcus folliculitis, Propionibacterium acnes, Propionibacterium avidum, Propionibacterium granulosum, Propionibacterium propionicus, Staphylococcus aureus, Staphylococcus auricularis, Staphylococcus capitis, Staphylococcus caprae, Staphylococcus cohnii, Staphylococcus epidermidis, Staphylococcus haemeolytica, Staphylococcus hominis, Staphylococcus lugdunensis, Staphylococcus massiliensis, Staphylococcus pettenkoferi, Staphylococcus warneri, Staphylococcus xylosus, Streptococcus dysgalactiae Streptococcus iniae, Streptococcus pneumoniae, Corynebacterium ienuis, Corynebacterium propinguum, Corynebacterium, flavescens, or Brevibacterium spp., or combinations thereof. The method includes applying to dermis of a human a biocompatible substrate comprising a natural fiber coated or embedded with one or more isolated phage with lytic activity specific for Micrococcus folliculitis, Propionibacterium acnes, Propionibacterium avidum, Propionibacterium granulosum, Propionibacterium propionicus, Staphylococcus aureus, Staphylococcus auricularis, Staphylococcus capitis, Staphylococcus caprae, Staphylococcus cohnii, Staphylococcus epidermidis, Staphylococcus haemeolytica, Staphylococcus hominis, Staphylococcus lugdunensis, Staphylococcus massiliensis, Staphylococcus pettenkoferi, Staphylococcus warneri, Staphylococcus xylosus, Streptococcus dysgalactiae, Streptococcus iniae, Streptococcus pneumoniae, Corynebacterium tenuis, Corynebacterium propinguum, Corynebacterium flavescens, or Brevibacterium spp., or combinations thereof.

Also provided is a method to prevent, inhibit or treat infection of the dermis by Streptococcus pyogenes, Serratia marcescens, Pseudomonas aeruginosa, Acinetobacter baumanni, Vibrio vulnificus, Bartonella spp., Pastuerella multocida, Capnocytophaga canimorsus, Klebsiella rhinoscleromatis, or Pseudomonas aeruginosa, or a combination thereof. The method includes applying to dermis of a human a biocompatible substrate comprising a natural fiber coated or embedded with one or more isolated phage with lytic activity specific for Streptococcus pyogenes, Serratia marcescens, Pseudomonas aeruginosa, Acinetobacter baumanni, Vibrio vulnificus, Bartonella spp., Pastuerella multocida, Capnocytophaga canimorsus, Klebsiella rhinoscleromatis, or Pseudomonas aeruginosa, or a combination thereof.

In one embodiment, the phage is delivered using a substrate comprising a non-natural polymer and optionally an adhesive, e.g., an adhesive backing. In one embodiment, the substrate comprises non-natural polymers, e.g., poly(glycolic acid), poly(anhydride), poly(amide), poly)ester), poly(orthoester), poly(lactide), poly(lactide-co-glycolide), poly(urethane), poly(ethylene), e,g., PEG, poly(acrylate), poly(caprolactone), or poly(oxazoline), e.g., poly(2-(diethylamino)ethyl methacrylate), 2-(dimethylamino)ethyl methacrylate), polyethylene glycol), poly(pyrrole), poly(amidoamine),poly(2-aminoethyl methacrylate), N-(2-hydroxypropyl)methacrylamide, poly(β-benzyl-1-aspartate), or poly(N-isopropylacrylamide), PLGA, or polyglutamic acid. In one embodiment, the substrate comprises Micrococcus folliculitis-targeting phage, Propionibacterium acnes-targeting phage, Propionibacterium avidum-targeting phage, Propionibacterium granulosum-targeting phage, Propionibacterium propionicus-targeting phage, Staphylococcus aureus-targeting phage, Staphylococcus auricularis-targeting phage, Staphylococcus capitis-targeting phage, Staphylococcus caprae-targeting phage, Staphylococcus cohnii-targeting phage, Staphylococcus epidermidis-targeting phage, Staphylococcus haemeolytica-targeting phage, Staphylococcus hominis-targeting phage, Staphylococcus lugdunensis-targeting phage, Staphylococcus massiliensis-targeting phage, Staphylococcus pettenkoferi-targeting phage, Staphylococcus warneri-targeting phage, Staphylococcus xylosus-targeting phage, Streptococcus dysgalactiae-targeting phage, Streptococcus iniae-targeting phage, Streptococcus pneumoniae-targeting phage, Corynebacterium tenuis-targeting phage, Corynebacterium propinguum-targeting phage, Corynebacterium flavescens-targeting phage, Brevibacterium spp.-targeting phage, Propionibacterium acnes-targeting phage, Staphylococcus epidermidis-targeting phage, or Staphylococcus aureus-targeting phage, or any combination thereof. In one embodiment, the substrate comprises a backing layer having composed of a beige flexible film with a low-density pigmented polyethylene outer layer and a polyester inner layer, a middle layer containing an adhesive, e.g.,a polyisobutylene/polybutene adhesive, and optionally one or more of crospovidone, non-woven polyester fabric and lauryl lactate as inactive components, and a composition comprising one or more different phage, as described above, and a third layer which protects the adhesive layer during storage and is removed just prior to application. In one embodiment, the third layer comprises a transparent polyethylene terephthalate (PET) film with a polydimethylsiloxane coating on the side that is in contact with the middle adhesive layer.

### DETAILED DESCRIPTION

### Definitions

As used herein, the term “isolated” in the context of phage refers to a phage which is separated from other molecules which are present in the natural source of the phage.

The term “purified” with respect to a bacteriophage means that the phage has been measurably increased in concentration by any purification process, including but not limited to, isolation from the environment or culture, e.g., isolation from culture following propagation and/or amplification, centrifugation, etc., thereby partially, substantially, nearly completely, or completely removing impurities, such as host cells and host cell components.

As used herein, the terms “prophylactic agent” and “prophylactic agents” refer to an agent, such as a bacteriophage cocktail, which can be used in the prevention, management, or control of one or more symptoms of a disease or disorder.

As used herein, the terms “therapeutic agent” and “therapeutic agents” refer to an agent, such as a bacteriophage cocktail that can be used in the treatment, management, or control of one or more symptoms of a disease or disorder.

As used herein, the terms “treat”, “treatment” and “treating” refer to obtaining a therapeutic benefit in a subject receiving a pharmaceutical composition. With respect to achieving a therapeutic benefit, the object is to eliminate, lessen, decrease the severity of, ameliorate, or slow the progression of the symptoms or underlying cause (e.g., bacterial infection) associated with the pathological condition or disorder. A “therapeutically effective amount” refers to that amount of a therapeutic agent, such as a phage cocktail pharmaceutical composition of the invention, sufficient to achieve at least one therapeutic benefit in a subject receiving the composition.

As used herein, the terms “prevent”, “prevention” and “preventing” refer to obtaining a prophylactic benefit in a subject receiving a pharmaceutical composition. With respect to achieving a prophylactic benefit, the object is to delay or prevent the symptoms or underlying cause (e.g., bacterial infection) associated with the pathological condition or disorder. A “prophylactically effective amount” refers to that amount of a prophylactic agent, such as a phage cocktail composition of the invention, sufficient to achieve at least one prophylactic benefit in a subject receiving the composition.

As used herein, the terms “antibacterial activity” and “antimicrobial activity”, with reference to a bacteriophage (or variant or fragment thereof) or bacteriophage product, are used interchangeably to refer to the ability to kill and/or inhibit the growth or reproduction of a microorganism, in particular, the bacteria of the species or strain that the bacteriophage infects.

### Phage Therapy

Bacteriophages, or phages for short, usually have a narrow host range. During infection of a bacterium, a phage has two principal life cycles it can enter—the lytic cycle and the lysogenic cycle. Both cycles are initiated by the attachment of the phage to a surface structure, which usually is species- and even strain-specific. After attachment, the phage injects its genetic material, which could be either DNA or RNA. After this injection, the phage can enter several different life cycles, with the lytic and lysogenic life cycle being the most common.

While all phages are capable of entering the lytic cycle (virulent phages), some phages (temperate phages) can also enter the lysogenic cycle. The lytic cycle results in production of phage particles; at the end of the cycle, the lysis cassette of the phage is expressed, producing a holin and a lysin. The holin forms a pore in the inner membrane, enabling the peptidoglycan-degrading lysin to get access to its target; this results in bacterial lysis and eventually in the release of new mature phage progeny.

### Exemplary Compositions and Methods

Thus, phages are viruses with the capacity of infecting bacteria in a very specific manner. Specifically, lytic phages are a class of phage that, through infection of host bacteria, are capable of decreasing populations of its host (target) bacteria without affecting other non-target bacterial strains. For example, P. acnes, S. epidermidis and S. aureus may be linked to acne infections. By using more than one phage isolate or strain with a different specificity, resistance to the combination of phage is unlikely. In one embodiment, phages are employed that bind to different receptors (e.g., using a phage cocktail), to lower the risk of developing resistance. Human skin surfaces are rich microbial environments and imbalances in the microbial ecology in these environments have a potential impact on skin appearance. In the present disclosure, the choice of the lytic phage specific for certain strains of bacteria will decrease the population of target bacteria and mitigate the corresponding skin appearance impact. Thus, phage can be employed to treat skin based disorders. The phage is administered using, for instance, natural fiber masks or patches, as a delivery vehicle. In one embodiment, lytic phage are embedded in a natural fiber mask/patch cloth. By applying this mask/patch to human surfaces, one can expose the microbial ecology to the bacteriophage.

In one aspect, the invention is directed to compositions comprising phage cocktails. In some embodiments, the invention provides compositions comprising at least two different isolated strains of phage. In another aspect, the instant invention is directed to compositions comprising phage cocktails and a pharmaceutically acceptable carrier. In some embodiments, the pharmaceutical composition is formulated for topical application. In some embodiments, the pharmaceutical composition comprises a sterile buffer, e.g., a buffer comprising about 0.05 M Tris-HCl, about 0.1M NaCl, and about 10 mM MgSO₄. In some embodiments, the composition further comprises an additional agent, e.g., an agent selected from the group consisting of an antibiotic agent, an anti-inflammatory agent, an antiviral agent, a local anesthetic agent, and a corticosteroid. In some embodiments, the additional agent is an antibiotic agent having antibacterial activity against bacteria other than P. acnes, S. epidermidis or S. aureus. In some embodiments, the composition is for use in treating a bacterial infection, and each of the phage strains is present in the composition in an amount corresponding to 10³ to 10¹³ phage particles/cm² of the area. In some embodiments, each of the phage strains is present in the composition in an amount corresponding to 10⁷ to 10⁹ phage particles/cm² of the area. Another aspect of the instant invention is directed to methods of treating or preventing a bacterial infection in a subject in need thereof comprising administering to the subject a therapeutically effective amount of the composition according to the invention. In some embodiments, the subject is a mammal, e.g., a human. In some embodiments, the treatment comprises topically administering the composition.

In one aspect, the invention relates to cocktail compositions of different phage strains. The “cocktail” may comprise at least two different isolated strains of phage, for example, two, three, four, five, six, seven, eight, nine, ten, or more different isolated bacteriophage strains. The cocktail may be used alone or in further combination with other therapies, e.g., antibiotic agents and/or growth factors. In some embodiments, the phage cocktail comprises at least 2 phage strains, at least 3 phage strains, at least 4 phage strains, at least 5 phage strains, at least 6 phage strains, at least 7 phage strains, at least 8 phage stains, at least 9 phage strains, at least 10 phage strains, or more. In some embodiments, the phage cocktail comprises 2-20 phage strains, 2-15 phage strains, 2-10 phage strains, 3-8 phage strains, or 4-6 phage strains. In more embodiments, the combination does not impair or reduce (or does not substantially or significantly impair or reduce) infecting ability and/or lytic activity of the individual bacteriophage in the presence of distinct bacteriophage strains

The phage cocktails of the present invention are incorporated into a composition for the use in topical treatment and/or prevention of bacterial infections. A cocktail of different phage strains, e.g., as disclosed herein, may be combined with a pharmaceutically acceptable carrier, such as an excipient or stabilizer. Examples of pharmaceutically acceptable carriers, excipients, and stabilizers include, but are not limited to, buffers such as phosphate, citrate, and other organic acids; antioxidants including ascorbic acid; low molecular weight polypeptides; proteins, such as serum albumin and gelatin; hydrophilic polymers such as polyvinylpyrrolidone; amino acids such as glycine, glutamine, asparagine, arginine or lysine; monosaccharides, disaccharides, and other carbohydrates including glucose, mannose, or dextrins; sugar alcohols such as mannitol or sorbitol; salt-forming counterions such as sodium.

The bacteriophage cocktail compositions of the present invention may also be combined with one or more non-phage therapeutic and/or prophylactic agents, useful for the treatment and/or prevention of bacterial infections, as described herein and/or known in the art (e.g. one or more antibiotic agents). Other therapeutic and/or prophylactic agents that may be used in combination with the phage cocktails of the invention include, but are not limited to, antibiotic agents, anti-inflammatory agents, antiviral agents, and corticosteroids. In some embodiments, the phage cocktail is administered in the absence of a non-phage based antibiotic agent.

Standard antibiotics that may be used with pharmaceutical compositions comprising a phage cocktail of the invention include, but are not limited to, amikacin, gentamicin, kanamycin, neomycin, netilmicin, paromomycin, rhodostreptomycin, streptomycin, tobramycin, apramycin, rifamycin, naphthomycin, mupirocin, geldanamycin, ansamitocin, carbacephems, imipenem, meropenem, ertapenem, faropenem, doripenem, panipenem/betamipron, biapenem, PZ-601, cephalosporins, cefacetrile, cefadroxil, cefalexin, cefaloglycin, cefalonium, cefaloridine, cefalotin, cefapirin, cefatrizine, cefazaflur, cefazedone, cefazolin, cefradine, cefroxadine, ceftezole, cefaclor, cefonicid, cefprozil, cefuroxime, cefuzonam, cefmetazole, cefotetan, cefoxitin, cefcapene, cefdaloxime, cefdinir, cefditoren, cefetamet, cefixime, cefmenoxime, cefteram, ceftibuten, ceftiofur, ceftiolene, ceftizoxime, ceftriaxone, cefoperazone, ceftazidime latamoxef, cefclidine, cefepime, cefluprenam, cefoselis, cefozopran, cefpirome, cefquinome, flomoxef, ceftobiprole, azithromycin, clarithromycin, dirithromycin, erythromycin, roxithromycin, aztreonam, pencillin and penicillin derivatives, actinomycin, bacitracin, colistin, polymyxin B, cinoxacin, flumequine, nalidixic acid, oxolinic acid, piromidic acid, pipemidic acid, rosoxacin, ciprofloxacin, enoxacin, fleroxacin, lomefloxacin, nadifloxacin, norfloxacin, ofloxacin, pefloxacin, rufloxacin, balofloxacin, gatifloxacin, grepafloxacin, levofloxacin, moxifloxacin, pazufloxacin, sparfloxacin, temafloxacin, tosufloxacin, clinafloxacin, garenoxacin, gemifloxacin, stifloxacin, trovalfloxacin, pnilifloxacin, acetazolamide, benzolamide, bumetanide, celecoxib, chlorthalidone, clopamide, dichlorphenamide, dorzolamide, ethoxyzolamide, furosemide, hydrochlorothiazide, indapamide, mafendide, mefruside, metolazone, probenecid, sulfacetamide, sulfadimethoxine, sulfadoxine, sulfanilamides, sulfamethoxazole, sulfasalazine, sultiame, sumatriptan, xipamide, tetracycline, chlortetracycline, oxytetracycline, doxycycline, lymecycline, meclocycline, methacycline, minocycline, rolitetracycline, methicillin, nafcillin, oxacilin, cloxacillin, vancomycin, teicoplanin, clindamycin, co-trimoxazole, flucloxacillin, dicloxacillin, ampicillin, amoxicillin and any combination thereof in amounts that are effective to additively or synergistically enhance the therapeutic and/or prophylactic effect of a phage cocktail of the invention for a given infection.

The compositions of the invention are administered topically (e.g., in the form of a lotion, solution, gel, cream, ointment, or powder on a substrate). Topical formulations generally include a sterile buffer, such as a sterile PBS, water, or saline buffer. One particular buffer comprises Tris-HCl, NaCl, and/or MgSO₄7H₂O, e.g., about 0.05 M Tris-HCl (pH 7.4-7.5), about 0.1 M NaCl, and/or about 10 mM MgSO₄7H₂O. In other embodiments, the formulation further comprises a buffer and 10 mM MgCl₂. In other embodiments, the phage containing formulation further comprises a buffer having about 5 mM to about 15 mM CaCl₂, e.g., about 10 mM CaCl₂.

In some embodiments, topical compositions, e.g., those having a substrate which is coated or embedded with a phage containing solution, are provided in a hermetically sealed container.

Exemplary Phage for Use with a Natural Fiber Substrate

Exemplary staphylococcal lytic phages include but are not limited to K, CS1, DW2, φSA012, φSA039; and Stau2 (Hsieh et al., Appl. Environ. Microbiol. 71:756 (2011)), as well as double-stranded DNA phages belonging to the Siphoviridae family of the Caudovirales order, e.g., philPLA35 and philPLA88 or SA11; Podoviridae and Myoviridae phages (Deshorian and Van Melderen, Viruses, 4:3316 (2012)), phi187, phi69, phi53, phi85, phi2638A, phi77, phi42e, phi3A, phi47, phi37, phiEW, phi96, phiROSA, phi71, phi55, phi29, phi52A, phi88, phiX2, phi92, phiPVL, phiSLT, phiPV83-pro, phi108-PVL, phiSa2mw, phiSa2958, phiSa2usa, phi7247PVL, phi5967PVL, phiETA, phiN315, phiMu50A, phiMu50B, phiSa3mw, phiSa3ms, phiSa3, phi11, phi12, phi13, L54a^{f}, phiNM1, phiNM2, phiNM3, phiNM4, phi80, phi80alpha, phiMR11, phiMR25, phiIPLA88, phiIPLA35, TEM126, SA11, 66, 44AHJD, P68, SAP2, K, G1, Twort, SAP1, SAP3, MSA6, phi812, SK311, Stau2, phiSA012, phiSA039, GH15, A5W, Staph1N, Fi200W, P4W, 676Z, A3R, ISP, Sb-1, or PT1028.

Exemplary phage specific for S. aureus include but are not limited to S. aureus bacteriophage vB_SauS-phi-IPLA88 (phiIPLA88) (Rodriquez-Rubio et al, PLOS One, 8:e64671 (2013)).

Exemplary phage specific for S. epidermidis include but are not limited to Podoviridae phages and Siphoviridae phages (Aswani et al., Appl. Enfviron. Microbiol, 77:7853 (2011)) as well as vB_SepiS-phiIPLA5 and vB_SepiS-philPLA7 (Guttierrez et al., BML Genomics, 13:228 (2012)) and double-stranded DNA phages belonging to the Siphoviridae family of the Caudovirales order, e.g., philPLA35 and philPLA88 or SA11; Podoviridae and Myoviridae phages (Deshorian and Van Melderen, Viruses, 4:3316 (2012)).

Exemplary phage specific for P. acnes include but are not limited to PA6 (Farrar et al., J. Baceriol., 189:4161 (2007)), P1.1; P9.1; P14.4; P100A; P100D; P100.1; P101A; P104A; P105; ATCC 29399B_C; ATCC29399B_T (Marinelli et al., mBIO, 3:4e00279 (2012)), PAD20 and PAS50 (Load and Collin, BMC Genomics, 12:198 (2011)), and accession numbers JX570702-JX570714, KJ578758-KJ578792 (Liu et al., ISME J., pp. 1-16 (2015)).

### Exemplary Formulations

The phage containing composition may be formulated in a variety of product forms, such as, for example, a lotion, cream, spray, aerosol, aqueous buffer solution, gel, towelette, mask, foam, and the like, for topical administration. In one embodiment, the phage are formulated as an aqueous solution or gel. The composition may include water; esters, isopropyl myristate and isopropyl palmitate; ethers such as dicapryl ether and dimethyl isosorbide; alcohols such as ethanol and isopropanol; fatty alcohols such as cetyl alcohol, cetearyl alcohol, stearyl alcohol and biphenyl alcohol; isoparaffins such as isooctane, isododecane and is hexadecane; silicone oils such as cyclomethicone, dimethicone, dimethicone cross-polymer, polysiloxanes and their derivatives, e.g., organomodified derivatives; polyols such as propylene glycol, glycerin, butylene glycol, pentylene glycol and hexylene glycol; or any combinations or mixtures of the foregoing. Aqueous vehicles may include one or more solvents miscible with water, including lower alcohols, such as ethanol, isopropanol, and the like.

In one embodiment of the invention, the composition may include additional skin actives such as, but are not limited to, botanicals, other keratolytic agents, desquamating agents, keratinocyte proliferation enhancers, collagenase inhibitors, elastase inhibitors, depigmenting agents, anti-inflammatory agents, steroids, anti-acne agents, antioxidants, thiodipropionic acid or esters thereof, and advanced glycation end-product (AGE) inhibitors. Exemplary anti-aging components include, without limitation, botanicals (e.g., Butea Frondosa extract); thiodipropionic acid (TDPA) and esters thereof; retinoids (e.g., all-trans retinoic acid, 9-cis retinoic acid, phytanic acid and others); hydroxy acids (including alpha-hydroxyacids and beta-hydroxyacids), salicylic acid and salicylates; other exfoliating agents e.g., glycolic acid, 3,6,9-trioxaundecanedioic acid, etc.), estrogen synthetase stimulating compounds (e.g., caffeine and derivatives); compounds capable of inhibiting alpha-reductase activity (e.g., linolenic acid, linoleic acid, finasteride, and mixtures thereof); barrier function enhancing agents (e.g., ceramides, glycerides, cholesterol and its esters, alpha-hydroxy and omega-hydroxy fatty acids and esters thereof, etc.); collagenase inhibitors; and elastase inhibitors; to name a few. In one embodiment, the composition comprises N-Acetyl Tyrosinamide.

Exemplary retinoids include, without limitation, retinoic acid (e.g., all-trans or 13-cis), and derivatives thereof, retinaldehyde, retinol (Vitamin A) and esters thereof, such as retinol palmitate, retinol acetate and retinol propionate, and salts thereof. Particular mention may be made of retinol. The retinoids will typically be included in amounts from about 0.0001% to about 5% by weight, more typically from about 0.01% to about 2.5% by weight or from about 0.1% to about 1.0% by weight. Compositions according to this embodiment will typically include an antioxidant such as ascorbic acid and/or BHT.

In another embodiment, the topical compositions of the present invention may also include one or more of the following: a skin penetration enhancer, an emollient, a humectant, a skin plumper, an optical diffuser, a sunscreen, an additional exfoliating agent, an antioxidant, and a pH adjuster.

A skin plumper serves as a collagen enhancer to the skin. An example of a suitable skin plumper is palmitoyl oligopeptide. Other skin plumpers are collagen and/or other glycosaminoglycan (GAG) enhancing agents. When present, the skin plumper may comprise from about 0.1 wt % to about 20 wt % wt the total weight in the composition.

Suitable exfoliating agents include, for example, alpha-hydroxyacids, beta-hydroxyacids, oxaacids, oxadiacids, and their derivatives such as esters, anhydrides and salts thereof. Suitable hydroxy acids include, for example, glycolic acid, lactic acid, malic acid, tartaric acid, citric acid, 2-hydroxyalkanoic acid, mandelic acid, salicylic acid and other derivatives thereof. An example of an exfoliating agent is glycolic acid. When present, the exfoliating agent may comprise from about 0.1 wt % to about 80 wt % of the composition.

An antioxidant functions, among other things, to scavenge free radicals from skin to protect the skin from environmental aggressors. Examples of antioxidants that may be used in the present compositions include compounds having phenolic hydroxy functions, such as ascorbic acid and its derivatives/esters; beta-carotene; catechins; curcumin; ferulic acid derivatives (e.g., ethyl ferulate, sodium ferulate); gallic acid derivatives (e.g., propyl gallate); lycopene; reductic acid; rosmarinic acid; tannic acid; tetrahydrocurcumin; tocopherol and its derivatives; uric acid; or any mixtures thereof. Other suitable antioxidants are those that have one or more thiol functions (—SH), in either reduced or non-reduced form, such as glutathione, lipoic acid, thioglycolic acid, and other sulfhydryl compounds. The antioxidant may be inorganic, such as bisulfites, metabisulfites, sulfites, or other inorganic salts and acids containing sulfur. In one particular embodiment, the inventive compositions will include TDPA or an ester thereof (e.g., dilauryl thiodipropionic acid), and/or an alpha hydroxyl acid (glycolic acid) and/or beta hydroxyl acid (salicylic acid or a derivative). Compositions of the present invention may comprise an antioxidant, which may comprise from about 0.001 wt % to about 10 wt %, or from about 0.01 wt % to about 5 wt %, of the total weight of the composition.

Other conventional additives include: vitamins, such as tocopherol and ascorbic acid; vitamin derivatives such as ascothyl monopalmitate; thickeners such as hydroxyalkyl cellulose; gelling agents; structuring agents; pigments; colorants; and pH adjusters. The composition may optionally comprise other components known to those skilled in the art including, but not limited to, film formers, moisturizers, minerals, viscosity and/or rheology modifiers, anti-acne agents, insect repellents, skin cooling compounds, skin protectants, lubricants, fragrances, preservatives, stabilizers, and mixtures thereof. In addition to the foregoing, the cosmetic compositions of the invention may contain any other compound for the treatment of skin disorders. The conventional additives, actives, adjuvants, and excipients set forth in the preceding paragraphs are present in the compositions in amounts suitable to obtain their intended purpose and effect, each typically being present in an amount of from 0.01 to 25% by weight of the cosmetic composition, in particular from about 0.1 to 5% by weight of the cosmetic composition.

The compositions may include liposomes. The liposomes may comprise other additives or substances and/or may be modified to more specifically reach or remain at a site following administration.

In one embodiment, the composition of the invention may have a pH between about 1 and about 8. In certain embodiments, the pH of the composition will be acidic, i.e., less than 7.0, for example, between about 2 and about 7, or between about 4.5 and about 7.5.

The compositions are applied to the skin for a period of time sufficient to inhibit bacterial growth on the skin. The compositions may be applied topically once, twice, or more daily. The composition may be applied to the skin one, two, or three times daily for as long as is necessary to achieve desired results. The treatment may be for a period of 15 minutes, 30 minutes, one hour or more. The treatment regiment may comprise daily application for at least one week, at least two weeks, at least four weeks, at least eight weeks, or at least twelve weeks. Chronic treatment regimens are also contemplated. In one embodiment, the phage are applied to the skin in an amount from about 10⁵ VLP/mL or 10⁷ VLP/mL to about 10¹⁰ VLP/mL, more typically from about 10⁶ VLP/mL to about 10⁹ VLP/mL, or from about 10⁸ VLP/mL, to about 10⁹ VLP/mL.

In one embodiment, the invention provides a composition and method for preventing, inhibiting or treating acne by topically applying a composition, e.g., in a cosmetically acceptable vehicle, over a select or an affected area for a period of time sufficient to reduce (inhibit) or prevent bacterial growth.

In one embodiment, the substrate may optionally be associated with a support, in particular a support which allows it to be held in place on the skin. The substrate in the form of a dry or moist sheet may be in the form of a mask or patch adapted to cover the face, or the contour of the cheeks, forehead or chin. Alternatively, the substrate in the form of a film may be associated with a flexible support with the aim of allowing easy application of the composition to the skin, for example in the form of a patch or a mask.

In one embodiment, the substrate comprises biocellulose. Biocellulose is a material obtained by aerobic fermentation, in an aqueous nutrient medium, of bacteria of the genus Acetobacter (also known as Gluconacetobacter) (Gromet-Elhanan, S. Hestrin, Synthesis of cellulose by Acetobacter Xylinum, J. Bacteriol., 85:284 (1963)). Biocellulose may be used in the pure form or in a form which is combined with other types of fibers, for example fibers of natural origin, for example fibers from corn (maize), hemp, linseed, cotton, jute, kenaf, raffia, ramie, toquilla, sisal, rush, alfalfa, phormium, coir, wool, silk, soya, Manilla hemp, kumazasa, persimmon, kapok, burdock, cereals, or bamboo. The biocellulose fibers may be free or bonded together and/or bonded to other fibers. The biocellulose may be used in sheet form obtained, for example, by compacting a culture of biocellulose fibers after washing them. The thickness of the sheet is in the range 50 μm to 4000 μm, for example. In one embodiment, the biocellulose based substrate may also contain 1% or 10% up to 99% of water, 40% to 90%.

In one embodiment, the substrate is sterilized before phage application, and after phage application, packaged in single-use packaging. Sterilization may be accomplished using means known to the skilled person.

The substrate may be packaged in the dry state for extemporaneous rehydration with water or other aqueous solution. By way of example, the other solution may contain at least one active ingredient, for example an active ingredient that is not incompatible with phage.

### Exemplary Fiber Layers

The substrate may be a fiber layer, e.g., a fiber layer constituted using a non-elastomer raw material and having elongatability at least in one direction, e.g., cotton, eucalyptus or biocellulose. For example, in one embodiment, a non-elastomeric fiber layer means a fiber layer having non-elastomeric properties and elongatability at least in one direction, in other words, a fiber layer having no elastomeric properties (rubber-like elasticity), but having elongatability at least in one direction. In one embodiment, the non-elastomeric fiber layer includes a fiber layer having elongatability but having a low recovery ratio after elongation. Such a fiber layer may be integrated through lamination with an elastomer layer, thereby moderate stretchability and a moderate stress during elongation being provided for a resulting cosmetic facial mask.

As the fiber layer, paper, a nonwoven fabric, a woven fabric and so forth can be utilized. In view easy handling and good texture when the layer is used in the form of a cosmetic facial mask, the fiber layer may be in the form of a nonwoven fabric. As the fibers constituting the fiber layer, for example, natural fibers such as pulp, cotton, linen, bamboo, kenaf, silk and wool, regenerated fibers such as viscose rayon, copra and lyocell, that may be combined with synthetic fibers such as polyethylene fibers, polypropylene fibers, polyethylene terephthalate fibers, polybutyrene terephthalate fibers, nylon fibers or polyacrylic fibers. Two or more kinds of fibers may be mixed and used. For example, a fiber blend of viscose rayon fibers with pulp, a fiber blend of viscose rayon fibers with cotton, a fiber blend of viscose rayon fibers with polyethylene terephthalate fibers, and a fiber blend of cotton with polyethylene terephthalate fibers can be utilized.

The fiber layer is hydrophilic and may have liquid retention properties. The fiber layer may constituted of hydrophilic fibers obtained using a hydrophilic raw material, and from non-elastomer raw material or the fibers formed therefrom materials generally evaluated to have hydrophilicity can be selected and used. In view of hydrophilicity, a raw material (cellulosic raw material) mainly formed of cellulose such as cotton, linen, rayon, copra and pulp may be used. A nonwoven fabric containing one kind or more kinds of raw materials with hydrophilicity, water-absorbing properties and liquid retention properties, can be used in the form of a cosmetic facial mask.

The fibers constituting the fiber layer may be short fibers or long fibers. For example, short fibers includes fibers having a fiber length of 0.5 to 100 mm, and long fibers includes fibers having a fiber length exceeding 100 mm, and include continuous fibers. The fiber diameter of the fibers constituting the fiber layer is not particularly limited, but may be about 0.1 to about 100 μm, e.g., about 4 to about 50 μm.

To the fiber layer, various modifiers such as a stabilizer, a non-phage antibacterial agent, a coloring agent, a lubricant, a hydrophilic agent, an antistatic agent, a charging agent, a slipping agent and/or an antiblocking agent, may be added.

### Optional Elastomer Layer

The substrate may include an elastomer layer, e.g., a layer structurally exhibiting elastomeric properties as derived from a fiber form or a layer constitution, or a layer produced using an elastomer raw material having elastomeric properties in a raw material itself. In order to secure desired stretchability, fitting capability, a skin tightening effect or a lift-up effect in the form of a cosmetic facial mask, the elastomer layer may be 50% or more in the “elongation recovery ratio at 50% elongation in a wet state,” e.g., 65% or more.

The elastomer layer can be used in a film shape, a sheet form, a net shape, a nonwoven fabric shape, a woven fabric shape or the like, as long as the layer has elastomeric properties as described above. In view of easy handling, texture softness, air permeability and liquid permeability when the layer is used in the form of a facial mask, and also excellent cost performance, the nonwoven fabric shape may be used.

The layer structurally exhibiting elastomeric properties as derived from a fiber form or a layer constitution is not particularly limited, and specific examples include a web containing short fibers provided with solid crimps, and a nonwoven fabric obtained by three-dimensionally entangling the fibers by a needle punch method, a water stream entangling method or a high-pressure steam entangling method, and also a woven fabric provided with stretchability by a processing method such as a stockinet stitch, a garter stitch and a rib stitch. Moreover, the layer may include a layer provided with stretchability by drawing processing using at least a pair of rolls in which tooth grooves are engaged. Moreover, the layer may be a layer including, in at least one layer, a nonwoven fabric prepared by using a web containing potentially crimpable fibers and heating the web to develop crimps of fibers and thereby entangle the fibers to unify the web.

When the elastomer layer includes a layer produced using an elastomer raw material having elastomeric properties in the raw material itself, specific examples of the elastomer raw materials to be used include various kinds of rubber such as natural rubber, chloroprene rubber, nitrile rubber, ethylene-propylene rubber, urethane rubber and silicone rubber, and thermoplastic elastomers such as a styrene-based elastomer, an olefin-based elastomer, a polyurethane-based elastomer, polyester-based elastomer, a polyamide-based elastomer and a vinyl chloride-based elastomer. In view of the fitting feeling associated with flexibility or stretchability of the cosmetic facial mask impregnated with a cosmetic liquid, or the like, a styrene-based elastomer, an olefin-based elastomer or a urethane-based elastomer may be employed. In order to appropriately adjust performance such as the stretchability in the elastomer layer, two or more kinds of different fibers may be mixed in the elastomer layer. The fiber diameter of fibers constituting the elastomer layer is not particularly limited, but may be in the range of 15 μm or less, e.g., in the range of 10 μm or less in order for the cosmetic facial mask to have desired flexibility and fitting capability onto the skin. The fibers constituting the elastomer layer may be short fibers or long fibers.

As the elastomer layer, a nonwoven fabric formed of long fibers as produced by a spunbond method or a melt-blown method using the thermoplastic elastomer raw material may be used in view of air permeability, liquid permeability, stretchability, and also cost performance. The elastomer layer can be produced by integrating, through lamination, a nonwoven fabric obtained by the spunbond method and a nonwoven fabric obtained by the melt-blown method.

In the elastomer layer, various modifiers such as a hydrophilic agent, a lipophilic agent, a slipping agent and an antiblocking agent, or a non-elastomeric raw material such as polyethylene, polypropylene, polystyrene, polyethylene terephthalate and nylon, may be added.

### Facial Mask

A facial mask may be manufactured from a natural fiber or using a laminate in which the fiber layer and the elastomer layer are integrated through lamination. Specifically, the mask may obtained by cutting out, from the laminate, a desired shape in a manner such that the direction of the laminated non-elastomeric fiber layer having elongatability, i.e., the direction of the laminate having stretchability, coincides with the vertical direction of face in the cosmetic facial mask.

When the fiber layer includes a fiber layer obtained by aligning fibers in one direction by carding, the cosmetic facial mask may be cut out in a manner such that the alignment of the fibers coincides with the cross direction of the cosmetic facial mask, i,e., the transverse direction of the face perpendicular to the vertical direction of the face.

A facial mask may be cut out into a shape suitable for covering the face, and processed. In the cosmetic facial mask, punched parts or cut parts are arranged, when necessary, for example, in parts corresponding to the eyes, the nose and the mouth. The processing method is not particularly limited, but punching using a Thompson blade or use of a rotary die cutter may be employed.

The facial mask cut out from a laminate has characteristics derived from the laminate, more specifically, a suitable stress at 50% elongation in the wet state, a suitable elongation recovery ratio at 50% elongation in the wet state, a suitable stress at 25% elongation recovery in the wet state, and a suitable basis weight, thickness or the like. Such a cosmetic facial mask has moderate stretchability and moderate elongation stress in the vertical direction of the face.

The facial mask may be appropriately cut out into various shapes depending on the purpose, and the shape is not particularly limited. For example, the mask may be of a one-piece type for wholly covering a face using one sheet, a separate type for wholly covering the face in combination of two or more sheets, or may have a shape of lengthening the neckline to cover a neck portion. Furthermore, the mask may have a shape for covering only a part of the face (the eyes, month, nose or cheek, for example). Moreover, the scope of the invention also includes a face mask having a constitution in which a member having stretchability in the vertical direction of the face is constituted to over apart a lift-up effect of which is desirably enhanced and have stretchability or elongatability in a direction and/or strength different from that of the above stretchability, or a member having neither elongatability nor stretchability covers other parts of the face.

In the facial mask, a good wearing feeling is obtained even without particularly making a cut, but for the purpose of more satisfactorily fitting the mask to the face, arbitrary number and arbitrary depth of cuts may be made in arbitrary position and direction of the cosmetic facial mask according to the shape of the cosmetic facial mask.

Furthermore, in a facial mask, any other raw material may be laminated (integrated through lamination) with at least part of the cosmetic facial mask within the range in which advantageous effects of the invention are not adversely affected. Although a site at which the lamination is made or any other raw material used for lamination is not particularly limited, a film, a woven fabric, a nonwoven fabric or the like may be used, and by selecting from a non-stretchable raw material, a stretchable raw material, a hydrophilic raw material, a non-hydrophilic raw material for a site at which efficacy is desirably locally obtained or the like depending on the use and the purpose, thereby allowing partial control of stretchability, partial control of liquid retention capability or partial provision of warmth retaining capability.

In the facial mask, the cosmetic liquid may be contained beforehand, immediately before use or during use. Moreover, it is possible to use a mask containing no cosmetic liquid as a wrap material after the cosmetic liquid is applied onto the face.

A mask prepared by coating a hydrous gel onto the substrate forming the cosmetic facial mask. The hydrous gel is not particularly limited, and specific examples thereof include agarose gel, sodium polyacrylate gel, bacterial cellulose gel and gelatin gel, etc.

In the cosmetic facial mask, the fiber layer, or the fiber layer and the elastomer layer may each independently, be present in a plurality of layers.

The invention will be further described by the following non-limiting example.

### EXAMPLE

The invention provides for packaging of phage or a combination of phage and delivery of phage to the epidermis to rebalance skin microbiomes for cosmetic purposes. In one embodiment, the phage are in solution (pH determined by the cocktail). The solution may contain one strain or any mixture of phage stains. The minimum titer level for a phage containing solution is about 10⁶ VLPs/mL up to about 10⁸ VLP/mL. In one embodiment, a phage concentration with a titer about 10⁸ VLPs/mL is prepared in an aqueous buffer. Fragrances or additives may be added to this solution. Reagents that inactivate phages, e.g., detergents, are not included in the solution. Any liquid additive that is a polar organic solvent (alcohol, aromatics) that is about <0.1% volume with a total of about <0.5% volume may be present. In one embodiment, a phage containing composition is contacted with a natural fiber substrate, e.g., cotton, eucalyptus, or biocellulose.

In one embodiment, a phage cocktail includes at least two of: Propionibacterium acnes targeting phage, Staphylococcus epidermidis targeting phage, Staphylococcus aureus targeting phage, or any combination thereof. In one embodiment, the buffer contains 0%-100% of Propionibacterium acnes targeting phage, 0%-100% of Staphylococcus epidermidis targeting phage, and 0%-100% of Staphylococcus aureus targeting phage, where the buffer has at least one of Propionibacterium acnes targeting phage, Staphylococcus epidermidis targeting phage, or Staphylococcus aureus targeting phage; has at least two of Propionibacterium acnes targeting phage, at least two of Staphylococcus epidermidis targeting phage, or at least two of Staphylococcus aureus targeting phage; or has at least one of each of a Propionibacterium acnes targeting phage, a Staphylococcus epidermidis targeting phage, and a Staphylococcus aureus targeting phage.

To prepare the delivery vehicle, in one embodiment, a high titer phage solution is produced, e.g., in an aqueous buffer, and a substrate, e.g., a cloth (mask), is immersed in the solution. In one embodiment, a dry mask, e.g., made of cotton, is added to the phage containing buffer for about >15 minutes. The buffer may also contain desirable fragrances or other aqueous chemicals. For example, in one embodiment, a mask surface that has no or reduced hydrophobic characteristics, is contacted with fragrances and/or oils, e.g., after contact with the phage containing solution. In one embodiment, the cloth (mask) may be treated with non-aqueous oils prior to soaking in the aqueous buffer. The mask is removed from the solution, and then introduced into packaging, e.g., an aluminum pouch, and sealed. Shelf life for phage activity may be at least 5 years. Alternatively, the phage containing substrate may be air dried under sterile conditions and packaged, e.g., in a pouch. In this embodiment, the substrate (or the skin area) needs to be wet in order to activate the phage. However, care needs to be taken not to wet the substrate in running water as the phage concentration may be effected (decreased). In one embodiment, the phages are suspended in an aqueous gel and the gel is sandwiched between two substrates, e.g., a natural fiber containing substrate and foil.

To apply, a sealed pouch is opened and the mask is applied to human skin, e.g., the face, for at least 10 minutes. The skin may be washed with water after application. Another wash with water about 12 hours post treatment may be useful to remove residual dead bacteria and excess phages.

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A composition for topical application, comprising:
a biocompatible substrate comprising a natural fiber coated or embedded with one or more isolated lytic phage with specificity for Propionibacterium acnes, for Staphylococcus epidermidis or for Staphylococcus aureus, or any combination thereof, in an amount effective to inhibit the growth of Propionibacterium acnes, Staphylococcus epidermidis or Staphylococcus aureus present on human dermis.

2. The composition of claim 1 wherein the natural fiber comprises cotton, eucalyptus, bamboo fiber, or biocellulose, or a combination thereof.

3. The composition of claim 1 wherein the one or more isolated phage are in an aqueous buffer.

4. The composition of claim 1 wherein the one or more isolated phage are in a gel.

5. The composition of claim 1 wherein the phage are embedded in the substrate.

6. The composition of claim 1 wherein the phage have a titer of at least 10⁶ VLP/mL or at least 10⁶ PFU/mL.

7. The composition of claim 1 wherein the natural fiber is coated or embedded with one or more strains of phage with specificity for P. acnes.

8. The composition of claim 1 wherein the natural fiber is coated or embedded with one or more strains of phage with specificity for S. aureus.

9. The composition of claim 1 wherein the natural fiber is coated or embedded with one or more strains of phage with specificity for S. epidermis.

10. The composition of claim 1 wherein the natural fiber is embedded with phage having specificity for P. acnes, phage having specificity for S. aureus, and phage having specificity for S. aureus.

11. The composition of claim 1 wherein the substrate comprises an adhesive backing.

12. A method to prepare a substrate to treat or inhibit bacterial growth on skin, comprising:
providing a biocompatible substrate comprising a natural fiber and an aqueous buffer or gel comprising one or more isolated lytic phage with specificity for Propionibacterium acnes, for Staphylococcus epidermidis or for Staphylococcus aureus, in an amount effective to inhibit or treat Propionibacterium acnes, Staphylococcus epidermidis or Staphylococcus aureus growth on human dermis; and
applying the aqueous buffer or gel to the substrate.

13. The method of claim 12 wherein the natural fiber comprises cotton, eucalyptus or biocellulose.

14. The method of claim 12 wherein the one or more phage are in an aqueous buffer.

15. The method of claim 12 wherein the one or more isolated phage are in a gel.

16. The method of claim 12 further comprising drying the substrate after applying the buffer or gel.

17. The method of claim 12 wherein the buffer is applied by immersing the substrate in the buffer.

18. The method of claim 12 wherein the natural fiber is embedded with phage having specificity for P. acnes, phage having specificity for aureus, and phage having specificity for S. aureus.

19. The method of claim 12 wherein the phage have a titer of at least 10⁶ VLP/mL or at least 10⁶ PFU/mL.

20. A method to prevent, inhibit or treat bacterial growth on skin, comprising applying to dermis of a human a biocompatible substrate comprising a natural fiber coated or embedded with one or more isolated lytic phage specific for Propionibacterium acnes, for Staphylococcus epidermidis or for Staphylococcus aureus, or a combination, thereof in an amount effective to inhibit or treat Propionibacterium acnes, Staphylococcus epidermidis or Staphylococcus aureus on the dermis.
